(19) United States
(12) Patent Application Publication
(10) Pub. No.: US 2019/0381072 A1
(43) Pub. Date: Dec. 19, 2019

(54) Preparation for the Prophylaxis and Treatment of Atypical Osteoporosis

(71) Applicant: Parapharm LLC, Penza (RU)

(72) Inventors: Villorij I. Strukov, Penza (RU); Olga Jones, Fort Worth, TX (US); Evgenij N. Krutiakov, Penza (RU); Konstantin G. Elistratov, Penza (RU)

(21) Appl. No.: 15/796,734

(22) Filed: Oct. 27, 2017

Related U.S. Application Data
(63) Continuation-in-part of application No. 14/395,478, filed on Mar. 9, 2015, now Pat. No. 9,827,273.

(30) Foreign Application Priority Data
Apr. 19, 2012 (RU) 2012115654

(51) Int. Cl. A61K 31/592; A61K 31/593

(52) U.S. Cl. CPC A61K 31/592; A61K 33/06; A61K 31/593

(57) ABSTRACT

A preparation for preventing and treating atypical osteoporosis with normal or increased bone tissue mineralization with the presence of cavities in trabecular bone sections, and conditions close to same, comprises between 10 mg and 1000 mg of drone brood and between 50 IU and 100,000 IU of vitamin D or vitamins of this group and/or the active metabolites in a daily dose. The preparation can be provided in powder, tablet or capsule form. It facilitates the redistribution of calcium in the body, reducing mineralization in soft tissues, vessels and other organs, as well as filling cavities in trabecular bone.

### CROSS-REFERENCE TO RELATED APPLICATIONS

The present application is a continuation-in-part of U.S. application Ser. No. 14/395,478 filed Aug. 21, 2012, pending, which is a U.S. National phase application of International application PCT/RU2012/000687 (publication WO 2013/157982) filed Aug. 21, 2012 and claiming priority from Russian application 2012115654 filed Apr. 19, 2012, all three above-identified applications being incorporated for reference into the present application in their entirety.

### BACKGROUND OF THE INVENTION

### 1. Field of the Invention

The invention relates to medicine and, in particular, to agents for the treatment and prevention of conditions associated with various forms of osteoporosis, specifically involving metabolic syndrome.

It has been known from the prior art that resulting from the discovery of D group vitamins, and, subsequently, of hormonal forms of vitamin D, the focus in treating osteopenia, osteoporosis and osteomalacia shifted to this group of preparations (Cholecalciferol (vitamin D₃), Ergocalciferol (vitamin D₂), Videchohun, Vigantol, Onealfa, Catcitriol, etc.). Calcium and vitamin D preparations have been combined to enhance calcium absorption. Medications such as ‘Calcium D₃ Nikomed’, ‘Calcemin Advance’, ‘Calcemin’, ‘Citrocal’, ‘Alphadol-Ca’ etc. are now widely used.

These agents suffer from the following disadvantages:

1) Preparations of calcium and vitamin D intensify activity of one another, and therefore there is a significant risk of overmineralizing various tissues and organs to the point of calcinosis, i.e. irreversible changes in the body of the patient. Uncontrolled use of calcium preparations may result in a drug-induced pathology, calcification of small and large vessels, formation of kidney stones, as well as stones in other organs.

2) The patient has already had contraindications, such as gall and kidney stones, hypercalcemia, etc. to the use of these agents.

Osteoporosis is a metabolic skeletal disease which is characterized by a reduction of bone mass per volume unit and by the microarchitectonics disorder of bone tissue, which lead to a reduction in the amount of calcium in the bones and to a high fracture risk for any bone including the hip.

The inventors established, and this has not been known from prior art, that deterioration and restoration of bone tissue mineralization happen unevenly. For instance, in postmenopausal osteoporosis bone density is first lost in trabecular sections and is then lost in cortical bone sections. Appropriate therapy can successfully cure osteoporosis, the restoration of the structure going in the reverse order and also unevenly. There are many clinical forms of osteoporosis, which can be systemic with a uniform bone lesion or with a primary lesion on separate parts of the skeleton such as vertebral bodies, limbs, etc. However, same treatment regimens are generally recommended for all forms of osteoporosis.

Consequently, an interest in hormonal mechanisms for regulating bone tissue mineral density has risen significantly.

It is known that D hormone ((active) metabolites of vitamin D) plays an important role in maintaining bone mineral density in both young and old men. Low level of D hormone (as well as low level of testosterone) is one of the causes of osteoporosis, namely, of the reduction of the bone mineral density in men, and, consequently, presents a risk factor for bone fractures.

Bisphosphonates are used to treat reduced bone density and osteoporosis. However, it has been proven that the effectiveness of these preparations is minimal if the D hormone and testosterone levels are low, because they provide for the proper absorption of the preparations aimed at restoring bone density. Therefore, treatment of bone density deterioration has to be complex and aimed both at restoring the deficiency of testosterone, if any, and at taking calcium preparations.

However, there are problems with the preparations (for example, testosterone propionate) that maintain the testosterone level in the body. The body produces even less of its own testosterone when testosterone is supplied to the body from an outside source.

The problem of increasing bone mineral density has been addressed with the aid of the agent ‘Osteomed’, a compound of calcium (between 10 wt. % and 95 wt. %) and drone brood (between 5 wt. % and 90 wt. %) (RU 2,412,616, A23L1/30, 2009). The introduction of drone brood is explained below. Drone brood is a donor of the following entomological sex hormones: prolactin, estradiol, progesterone and testosterone, which stimulate reproductive functions in men and women. Drone brood, which is saturated with hormones and vitamins that are not hormonal replacement, is efficient at endocrine profile imbalance and stimulates the central mechanisms regulating androgen formation intensity and eliminates the possibility of replacement therapy.

However, ‘Osteomed’ contains a calcium compound. There is much calcium in patients with hypermineralization, and calcium deposits have even been observed in their soft tissues (muscles). It is strongly recommended that these patients not take calcium preparations. However, the authors found that these patients have trabecular bone cavities which can cause fractures, particularly if they have a history of this problem. This result was found by the authors and was not previously known. Therefore, these patients should not take ‘Osteomed’. The above combination of factors will be further on referred to as atypical osteoporosis.

### SUMMARY OF THE INVENTION

The object of the claimed invention consists in creating an agent that can facilitate redistribution of calcium in the body: reducing mineralization in soft tissues, vessels and other organs, and being instrumental in the filling of trabecular bone cavities.

This object is achieved by providing a preparation for the prevention and treatment of atypical osteoporosis with normal or increased bone tissue mineralization with the presence of cavities in trabecular bone sections (and conditions close thereto involving excess mass and metabolic syndrome), which preparation comprises between 10 mg and 1000 mg of drone brood and between 50 IU and 100,000 IU of vitamin D or vitamins of group D and/or the active metabolites thereof and the preparation is provided in powder, tablet or capsule form.

The preparation was created and tested on volunteers, the ratio of ingredients being between 50 IU and 100,000 IU per day of vitamin D or vitamins of this group (and/or the active metabolites thereof); and between 10 mg and 1000 mg per day of drone brood. The claimed agent can be provided in powder, tablet or capsule form.

Vitamin D is included because although drone brood is saturated with vitamins, vitamin D3 being among others, but in non-replenishing, small doses. Therefore, the concentration thereof is insufficient for treating osteoporosis.

Drone brood has to be introduced as a donor of the following sex hormones which have a positive effect on bone mineralization: estradiol, progesterone and testosterone.

The range of the ingredients in the claimed preparation is determined by the patient demographics: age, eating habits, lifestyle, race, country of habitation, gender, and genetic and previous diseases. A doctor assessing these criteria selects the specific proportion of the ingredients and adjusts it based on cavity closure dynamics.

An explanation of the range limits:

1) Between 50 IU and 100,000 IU per day of vitamin or vitamins of group D (and/or active metabolites thereof), the lower limit being selected based on effectiveness, the upper limit—based on toxicity.

2) Between 10 mg to 1000 mg per day of drone brood, the lower limit being selected based on the effectiveness, the upper limit—based on the feasibility of use in terms of the ratio of effectiveness/price rise of the product.

The studies have established that using the claimed preparation strengthens the mechanism of the uniform restoration of bone mineral density, the use of drone brood in combination with vitamin D additionally aims at enhancing the remodeling of damaged bone tissue sections and bone tissue retention by maintaining androgen levels.

The combined use of vitamin D with drone brood makes it possible to achieve the greatest effectiveness in osteoporosis therapy in patients with hypercalcemic conditions and reduce the frequency of such adverse side effects as calcified deposits and stones forming in the kidneys and in other organs.

Although the components of the claimed preparation have, been known in folk and traditional medicine, the combination thereof in one product is not known, and it is just uncovered synergistic effect that makes it possible to solve the problem of balanced bone mineralization in both trabecular bone sections and cortical bone sections and achieve the object of the invention, eliminating or reducing the imbalance in the mineralization of various bone tissue sections.

Examples of closing cavities using the homogenate of drone brood and vitamin D3 in patients with hypermineralization:

Example No. 1: Patient FAD, female, 64, with postmenopausal osteoporosis. The patient was taking for a year Calcium D₃ Forte made by ‘NIKOMED’. She has been diagnosed with hypermineralization, salt deposits in her soft tissues. Despite this condition, the patient has cavities. She was prescribed treatment with the claimed agent in the form of a powder mixture: 1000 mg of drone brood+100 IU of vitamin D₃ per day. The picture taken at the beginning of treatment clearly showed salt deposits in soft tissues, which indicates hypermineralization. After nine months of treatment, it could be seen that the claimed agent reduced salt deposits and closed the cavities. In other words, calcium was redistributed within the body.

Example No. 2: Patient Z., female, 66, with postmenopausal osteoporosis. The patient was taking for nine months Calcium D₃ Forte made by ‘NIKOMED’. She has been diagnosed with hypermineralization, salt deposits in her soft tissues. Despite this condition, the patient has cavities. She was prescribed treatment with the claimed agent in a composition in the form of a powder mixture: 500 mg of drone brood+2500 IU of vitamin D3 per day. After six-month treatment, the cavities were reduced and the salt deposits in the soft tissues disappeared.

Example No. 3: Patient Z1, female, 70, with postmenopausal osteoporosis. The patient was taking for 12 months Calcium D₃ Forte made by ‘NIKOMED’. She has been diagnosed with hypermineralization, salt deposits in her soft tissues. Despite this condition, the patient has large cavities. She was prescribed treatment with the claimed agent in a composition in the form of a powder mixture: 500 mg of drone brood+100,000 IU of vitamin D2 per day for six months. The cavities closed, but the salt deposits in the soft tissues did not completely disappear, which shows that too large dose of vitamin D2 for this patient was used.

Regarding the range for using vitamin D and the active metabolites thereof, it should be noted that various forms of vitamin D have various degrees of therapeutic activity. Thus, vitamin D₂ is weaker than vitamin D₃, and therefore a greater amount of D₂ is required in comparison to vitamin D₃. The same is true regarding vitamin D metabolites.

Example No. 4, to show ineffectiveness of the claimed agent where its dosage is lower than the low limit. The patient D., female, 60, with postmenopausal osteoporosis. The patient was taking Calcium D₃ Forte, made by NIKOMED, for six months. She has been diagnosed with hypermineralization—salt deposits in her soft tissues. No cavities yet. She was prescribed treatment with the claimed agent in a composition in the form of a powder mixture: 9 mg of drone brood and 40 IU of vitamin D₂ per day for six months. As a result, there were no significant changes, the bone mineral density has been reduced, the mineral deposits have also proportionally reduced.

When prescribing the claimed agent, the doctor chooses the dosage of the ingredients thereof individually, based on the patient disease status. The condition of the patient is recommended to evaluate every 6-9 months using densitometry apparatuses to measure cavities and adjusting the specific composition of the claimed agent.

We claim:

1. A preparation for preventing and treating atypical osteoporosis with normal or increased bone tissue mineralization with the presence of cavities in trabecular bone sections, and conditions close thereto involving excess mass and metabolic syndrome, the preparation comprising in a daily dose between 10 mg and 1000 mg of drone brood and between 50 IU and 100,000 of vitamin D or vitamins of group D and/or the active metabolites thereof.

2. The preparation according to claim 1, wherein it is provided in powder, tablet or capsule form.
